(19) United States
(12) Patent Application Publication
(10) Pub. No.: US 2021/0137543 A1
(43) Pub. Date: May 13, 2021

(54) METHOD OF TREATING THE LUNGS

(71) Applicant: Tissue Regeneration Technologies, LLC, Woodstock, GA (US)

(72) Inventors: John F. Warlick, Woodstock, GA (US); John Patrick Finney, Hagerhill, KY (US)

(21) Appl. No.: 16/830,924

(22) Filed: Mar. 26, 2020

Related U.S. Application Data
(60) Provisional application No. 62/932,756, filed on Nov. 8, 2019.
(60) Provisional application No. 62/963,371, filed on Jan. 20, 2020.

(51) Int. Cl. A61B 17/22

(52) U.S. Cl. CPC A61B 17/22004; A61B 2017/00809

(57) ABSTRACT

A method of treating a patient exhibiting a lung disease or pulmonary disorder by applying shock waves or acoustic pulses directed to impinge lung tissue of the lung or lungs exhibiting a lung disease or pulmonary disorder, has the steps of: activating an acoustic shock wave or acoustic wave generator or source to emit acoustic shock waves or pressure pulses from a fixed acoustic wave source or a handheld shock wave or pressure pulse head; and administering a plurality of acoustic waves in a pressure pulse or shock wave pattern within the lung tissue of less than 10.0 mJ/mm2 per shock wave, the plurality of acoustic waves in a pressure pulse or shock wave pattern being directed to a portion of the lung exhibiting the lung disease or pulmonary disorder.

### TECHNICAL FIELD

The present invention relates to a treatment for lungs using acoustic shock waves, more particularly lungs exhibiting one or more diseases, including but not limited to Chronic Obstructive Pulmonary Disease (COPD).

### BACKGROUND OF THE INVENTION

Many people experience problems breathing. The main function of the lungs is the process of gas exchange called respiration or breathing. In respiration, oxygen from incoming air enters the blood and carbon dioxide, a waste gas from the metabolism, leaves the blood. A reduced lung function means that the ability of the lungs to exchange gases is reduced.

Diseases of the lungs inhibit the flow of oxygen into the blood stream which affects all the other body functions reducing the person's brain activity and physical stamina. Some common lung diseases include asthma, bronchitis, Chronic Obstructive Pulmonary Disease (COPD), cystic fibrosis, emphysema, Idiopathic pulmonary fibrosis (IPF), flu, lung cancer, obstructive sleep apnea, pleurisy, pneumonia, tuberculosis (TB).

Some of these diseases are treatable and can be cured, others simply can be controlled but not cured. Ideally, a cure for every type of lung disease would be possible.

The present inventive medical treatment brings the possibility to not only mitigate these diseases, but in many cases cure the patient in such a way that normal lung function is achieved.

The present inventors have been involved in the development of acoustic sound waves or pressure pulses over the last decade in the treatment of tissue and organs. They have led the medical community in a variety of breakthrough medical treatments for a variety of conditions. Recently, they discovered that pressure pulses or acoustic sound waves could, contrary to the common belief, be applied directly to the thin delicate tissue membranes of the lungs. Heretofore, those skilled in the art felt that directing such energy to the lungs would risk tearing or rupturing the lung. Shields and other devices were developed to prevent this from occurring. In particular, in treating the heart with sound waves, the inventors went to great trouble to avoid an emission path that would impinge the lung sacs.

Recently, one of these same inventors discovered a unique way to overcome these concerns and treat the diseases of the lungs directly using acoustic waves and pressure pulses without risk of damage to the lung tissue. These novel methods are described herein.

### SUMMARY OF THE INVENTION

A method of treating a patient exhibiting a lung disease or pulmonary disorder by applying shock waves or acoustic pulses directed to impinge lung tissue of the lung or lungs exhibiting a lung disease or pulmonary disorder, has the steps of: activating an acoustic shock wave or acoustic wave generator or source to emit acoustic shock waves or pressure pulses from a fixed acoustic wave source or a handheld shock wave or pressure pulse head; and administering a plurality of acoustic waves in a pressure pulse or shock wave pattern within the lung tissue of less than 10.0 mJ/mm2 per shock wave, preferably less than 1.0 mJ/mm2, the plurality of acoustic waves in a pressure pulse or shock wave pattern being directed to a portion of the lung exhibiting the lung disease or pulmonary disorder.

The step of administering a plurality of acoustic waves delivered as shock waves or pressure pulses to the lung further reduces symptoms of the lung disease or pulmonary disorder.

The lung disease or pulmonary disorder can be one or more of asthma, bronchitis, Chronic Obstructive Pulmonary Disease (COPD), cystic fibrosis, emphysema, Idiopathic pulmonary fibrosis (IPF), flu, lung cancer, obstructive sleep apnea, pleurisy, pneumonia, or tuberculosis (TB).

In one embodiment, the treatment further involves administering acoustic shock waves or pressure pulses directed to an area of the lung, or to a reflexology zone to treat the lung disease or pulmonary disorder, preferably to both.

The reflexology zone is at an extremity of a limb, preferably the extremity is a hand or foot.

The acoustic shockwave or acoustic wave generator or source can be a spherical, ballistic, radial, piezoelectric, electrohydraulic, electromagnetic or other similar device.

### Definitions

A “curved emitter” is an emitter having a curved reflecting (or focusing) or emitting surface and includes, but is not limited to, emitters having ellipsoidal, parabolic, quasi parabolic (general paraboloid) or spherical reflector/reflecting or emitting elements. Curved emitters having a curved reflecting or focusing element generally produce waves having focused wave fronts, while curved emitters having a curved emitting surfaces generally produce wave having divergent wave fronts.

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different ways, for example: A focused wave will become divergent once it has passed through the focal point. Spherical waves are also included in this definition of divergent waves and have wave fronts with divergent characteristics.

“Extracorporeal” occurring or based outside the living body.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plain waves. “Nearly plane” waves can be emitted by generators having pressure pulse/shock wave generating elements with flat emitters or curved emitters. Curved emitters may comprise a generalized paraboloid that allows waves having nearly plane characteristics to be emitted.

A “pressure pulse” according to the present invention is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nano-seconds (ns) up to some milli-seconds (ms). Very fast pressure pulses are called shock waves. Shock waves used in medical applications do have amplitudes above 0.1 MPa and rise times of the amplitude can be below 1000 ns, preferably at or below 100 ns. The duration of a shock wave is typically below 1-3 micro-seconds (μs) for the positive part of a cycle and typically above some micro-seconds for the negative part of a cycle. These typical time durations can be compressed by employing very high frequency devices of 1000 Hz or more while still maintaining a symmetric profile of a shock wave all of which are included within the scope of the present invention. In addition to the more common sources of shock wave or pressure pulse generators such as radial, spherical, electrohydraulic, piezoelectric and ballistic generators, the present invention contemplates laser generators. Laser generators produce numerous tiny acoustic waves as the laser beam pulses. The lower energy shock waves generated by lasers mimic the more conventional sources of sound waves and are therefore to be included herein.

“Shock Wave”: As used herein is defined by Camilo Perez, Hong Chen, and Thomas J. Matula; Center for Industrial and Medical Ultrasound, Applied Physics Laboratory, University of Washington, 1013 NE 40th Street, Seattle, Washington 98105; Maria Karzova and Vera A. Khokhlovab; Department of Acoustics, Faculty of Physics, Moscow State University, Moscow 119991, Russia; (Received 9 Oct. 2012; revised 16 Apr. 2013; accepted 1 May 2013) in their publication, “Acoustic field characterization of the Duolith: Measurements and modeling of a clinical shock wave therapy device”; incorporated by reference herein in its entirety.

Waves/wave fronts described as being “focused” or “having focusing characteristics” means in the context of the present invention that the respective waves or wave fronts are traveling and increase their amplitude in direction of the focal point. Per definition the energy of the wave will be at a maximum in the focal point or, if there is a focal shift in this point, the energy is at a maximum near the geometrical focal point. Both the maximum energy and the maximal pressure amplitude may be used to define the focal point.

Diseases of the lung and pulmonary disorders: these can have various causes such as diseases, viruses, microorganisms, bacteria, infections, etc.

Asthma is a chronic (life time) disease that makes your lungs very sensitive and hard to breathe. Asthma can't be cured, but with proper treatment, people with asthma can lead normal, active lives. If you have asthma, your airways (breathing passages) are very sensitive. Certain things can make your airways become: Swollen and filled with mucus—the swelling and mucus makes your airways narrower, so it is hard for air to pass through; Small and tight—your airways might also become twitchy and squeeze together and tighten. This makes your airways narrower and hard for air to pass through.

Bronchitis means swelling in your air passages (bronchi). Bronchi are the air passages that connect your windpipe (trachea) with tiny air sacs (alveoli) in your lungs. The air sacs are where your body absorbs the oxygen you breathe in. Bronchitis is an inflammation of the bronchi. This inflammation means the walls of your bronchi are swollen and filled with extra sticky mucus. Airflow into and out of your lungs is partly blocked because of the swelling and extra mucus in your bronchi. This makes you cough. There are two kinds of bronchitis: Acute bronchitis makes you sick for a while, but gets better after two to three weeks. Chronic bronchitis doesn't go away. With chronic bronchitis, you have a cough with mucus most days for three months of the year.

COPD means Chronic Obstructive Pulmonary Disease. It is a term that covers two types of chronic (long-term) diseases where the airways (breathing tubes) in the lungs become swollen and partly blocked. COPD gets worse over time. It cannot be cured, but it can be treated and managed. COPD consists of two major breathing diseases: emphysema and chronic bronchitis. Emphysema damages the tiny alveoli (air sacs) at the tips of your lungs. Normally these air sacs stretch like balloons as you breathe in and out. Emphysema makes these air sacs stiff. Because they cannot stretch, air gets trapped inside them. This makes it difficult for you to breathe in and makes you feel tired. Chronic bronchitis makes your airways red, swollen and irritated. Glands in your airways make extra mucus (phlegm), which blocks some air from passing through. This makes you cough, cough up mucus and feel short of breath. Many people with COPD have both of these diseases.

Coronavirus: A coronavirus is a type of common virus that can infect your nose, sinuses, or upper throat. They can spread much like cold viruses. Almost everyone gets a coronavirus infection at least once in their life, most likely as a young child. Most coronaviruses are not dangerous, but some are. Those that cause Middle East respiratory syndrome (MERS) or severe acute respiratory syndrome (SARS) can be deadly. Influenza (the flu) and COVID-19, the illness caused by the new coronavirus, are both infectious respiratory illnesses. Although the symptoms of COVID-19 and the flu can look similar, the two illnesses are caused by different viruses.

Cystic fibrosis (CF) Cystic fibrosis mainly affects people's lungs and digestion. People with cystic fibrosis have an unusually thick, sticky mucus that clogs their lungs, makes it hard to breathe, and can lead to life-threatening lung infections. CF also affects the pancreas: thick secretions there stop the release of the digestive enzymes that normally help break down food, making it hard for people to digest and absorb nutrients. The mucus can also block the bile duct in the liver, which eventually causes permanent liver damage in some people with CF.

Emphysema is a serious respiratory disease, which is another form of COPD. The most common cause is smoking. Those who suffer from emphysema have trouble exhaling air from their lungs. Cigarette smoke damages the air sacs in the lungs to a point where they can no longer repair themselves.

Idiopathic pulmonary fibrosis (IPF) is a type of lung disease that results in scarring (fibrosis) of the lungs for an unknown reason. Over time, the scarring gets worse and it becomes hard to take in a deep breath and the lungs cannot take in enough oxygen. IPF is a form of interstitial lung disease, primarily involving the interstitium (the tissue and space around the air sacs of the lungs), and not directly affecting the airways or blood vessels. There are many other kinds of interstitial lung disease that can also cause inflammation and/or fibrosis, and these are treated differently. It is important to work with your doctor to determine if you have IPF or another form of interstitial lung disease.

The flu is a highly contagious illness caused by the influenza virus. The influenza virus causes infections of the nose, throat and lungs. In most people, the flu is uncomfortable and tiring. It can keep people in bed for days or even a couple of weeks. Some people are more at risk for serious complications from the flu, including seniors, young children, and people with long-term lung diseases like asthma and chronic obstructive pulmonary disease (COPD). Flu can make asthma symptoms worse and cause COPD flare-ups. Like the regular flu, H1N1 (swine flu) can lead to more serious problems including pneumonia, a lung infection, and other breathing problems.

Hantaviruses are a family of viruses spread mainly by rodents and can cause varied disease syndromes in people worldwide. Infection with any hantavirus can produce hantavirus disease in people. Hantaviruses in the Americas are known as “New World” hantaviruses and may cause hantavirus pulmonary syndrome (HPS).

Lung cancer is cancer that starts in the lungs. Cancer is a disease where cancer cells grow out of control, taking over normal cells and organs in the body. There are two major types of lung cancer; non-small cell cancer and small cell lung cancer. Each type of lung cancer grows and spreads in different ways. Each type may be treated differently.

Pleurisy is an inflammation of the pleura. The pleura is a two layered membrane that both encloses the lung and lines the chest cavity. People have two pleurae, one around each lung. The pleurae act as a protective wrapping, fitting snugly over your lungs. Pleurae are made up of two layers. Normally, there is no space between the inner and outer layer. The layers are joined at the edges, so that the pleura might be compared to a balloon with no air, completely empty of air and wrapped tightly around the outside of each of the lungs. Normally, there is nothing but a thin layer of lubricating layer of fluid between the inner pleural lining and the outer pleural lining The smooth pleura linings and lubricating fluid allow your lungs to move freely in your chest, as they do in normal breathing. In people with pleurisy, the two layers of pleura get inflamed (red and swollen). This can create a space between the layers called the pleural cavity (cavity means space). In wet pleurisy, this space can fill up with fluid that can get infected.

Pneumonia (nu-MO-ne-ah) is swelling (inflammation) of one or both lungs that is usually caused by an infection. Many different germs can cause pneumonia, including bacteria, viruses, and fungi. When you breathe in these germs, they can settle in the air sacs (alveoli) of your lungs. Deep in your lungs, the germs may grow and overcome your body's normal defenses. After the lungs become infected, the air sacs (alveoli) in the lungs fill with pus and mucus. This swelling (inflammation) of the air sacs makes them less stretchy and keeps oxygen from properly reaching your blood stream.

Obstructive sleep apnea (also called OSA or obstructive sleep apnea-hypopnea syndrome) means you have short pauses in your breathing when you sleep. These breathing pauses—called apneas or apnea events—last for 10 to 30 seconds, maybe longer. People with obstructive sleep apnea can stop breathing dozens or hundreds of times each night leading to sleep disruption and low levels of oxygen.

Tuberculosis (TB) is a serious disease caused by breathing in a bacteria called Mycobacterium tuberculosis. TB usually infects the lungs. TB can also infect other parts of the body, including the kidneys, spine and brain.

### BRIEF DESCRIPTION OF THE DRAWINGS

The invention will be described by way of example and with reference to the accompanying drawings in which:

FIG. 1 is a simplified depiction of a pressure pulse/shock wave (PP/SW) generator with focusing wave characteristics.

FIG. 2 is a simplified depiction of a pressure pulse/shock wave generator with plane wave characteristics.

FIG. 3 is a simplified depiction of a pressure pulse/shock wave generator with divergent wave characteristics.

FIG. 4 is a simplified depiction of a pressure pulse/shock wave generator being connected to a control/power supply unit.

FIG. 5 shows an exemplary pressure pulse/shock wave generator device.

FIG. 6 shows a patient being treated extracorporeally with shock waves or pressure pulses being transmitted through the skin and spinal bone tissue to the respiratory region of the lung to be treated.

FIG. 7 shows a diagram of lungs.

FIG. 8 shows a patient being treated extracorporeally with shock waves or pressure pulses to a reflexology zone of the hand for the respiratory system.

FIG. 9 shows a patient being treated extracorporeally with shock waves or pressure pulses to a reflexology zone of the foot for the respiratory system.

### DETAILED DESCRIPTION OF THE INVENTION

All lung disease or pulmonary disorders can be effectively treated with shock waves by directly treating the lungs exhibiting inflammation or disease. Obviously, lung disease can be attributed to other things, such as a site specific injury; however, by way of example most respiratory function loss starts in the lung. This is often caused by radiating inflammation, the same as if you injure your shoulder and keep utilizing it until eventually your whole upper arm and neck hurts. In the case of the lungs, there is no way to truly rest and recover the lung tissue.

The use of acoustic shock waves or pressure pulses on the lungs has been found to thin the mucus in the lungs and allows the mucus to easily dislodge from the afflicted lung tissue. Patients cough up quantities of this thinned mucus material and recover breathing and respiratory performance due tot eh mechanical action generated by the pulsed acoustic shock wave or pressure pulse treatment. This is accomplished using mechanical forces as the primary action as opposed to the current practice of medication to thin the entrapped mucus and thereafter using an endoscope with mechanical suction to extract the mucus, this is often a procedure requiring the patient to be stable enough to be endoscoped. Many patients are too weakened to be treated in such an invasive fashion and this leads to their death. The present invention provides the treatment regardless of the level of seriousness of the patient's condition. This affords a new treatment to combat the mucus fluid entrapped in the respiratory system allowing the lungs to oxygenate the blood and recover breathing functions right after a treatment. This reduces the mortality rate of patients afflicted with respiratory illness or disease from virus, bacteria or other causes of pneumonia type symptoms. The use of mucus thinning medication can be combined with acoustic shock wave or pressure pulse treatments if desired and the use of endoscopic suction can also be performed in combination assuming the patient is strong enough, however, current studies indicate the use of these conventional treatments are not required, but are mentioned only as an option. Secondarily, the acoustic shock wave or pressure pulse treatment has the added benefit of germicidally eradicating the infection whether it is from bacterial or viral sources.

The goal in such treatments is to provide 100 to 3000 acoustic shock waves or pressure pulses at a voltage of 14 kV to 28 kV across a spark gap generator or a ballistic wave generator in a single treatment preferably or one or more adjuvant treatments by impinging the emitted waves on the lungs.

The unfocused shock waves or pressure pulses can be of a divergent wave pattern or near planar pattern preferably of a low peak pressure amplitude and density. Typically, the energy density values of the shock waves range as low as 0.000001 mJ/mm² and having a high-end energy density of below 1.0 mJ/mm², preferably 0.40 mJ/mm² or less, more preferably 0.20 mJ/mm2 or less. The peak pressure amplitude of the positive part of the cycle should be in the rage of nano-second up to some milliseconds and its duration is below 1-3 microseconds.

The pressure pulse is much slower, a “pressure pulse” according to the present invention is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nano-seconds (ns) up to some milli-seconds (ms).

The treatment depth can vary from the surface to the full depth of the human or animal torso above the lungs and the treatment site can be defined by a much larger treatment area than the 0.10-3.0 cm² commonly produced by focused waves. The above methodology is particularly well suited for the sub-surface soft tissue treatments of the lungs and more particularly the affected areas exhibiting the lung disease.

An exemplary treatment protocol could have emitted shock waves in a broad range of 0.01 mJ/mm² to 3.0 mJ/mm² and 200-2500 pulses per treatment with a treatment schedule of 1-3 weekly treatments until symptoms reduce. This can be repeated as symptoms reoccur or continue weekly as a preventative. The post medical treatment is beneficial as a disease suppressor and reduces the need for medications such as expensive asthma medications and allows less addictive medications to be used to prevent addiction.

The following invention description first provides a detailed explanation of acoustic shock waves or pressure pulses, as illustrated in FIGS. 1-6. As used herein an acoustic shock wave is an asymmetric wave with an exceptionally rapid peak rise time and slower return time from the peak amplitude. Historically, these acoustic shock waves or pressure pulses were first used medically to destroy kidney stones. The wave patterns were directed to a focal point with a relatively high energy to blast the concrements into small urinary tract passable fragments.

A whole class of acoustic shock waves or pressure pulses for medical treatments were later discovered that employed low energy acoustic shock waves or pressure pulses. These low energy acoustic shock waves or pressure pulses maintained the asymmetric wave profile, but at much lower energies as described in US2006/0100550 which is incorporated herein in its entirety.

These low energy acoustic shock waves or pressure pulses advantageously could stimulate a substance without requiring a focused beam. The advantage of such an unfocused beam was the acoustic wave could be directed to pass through tissue without causing any cell rupturing which would be evidenced by a lack of a hematoma or bruising. This use of unfocused, low energy acoustic shock waves or pressure pulses provided an ability to treat a large volume of tissue virtually painlessly. Furthermore, the acoustic energy caused a short duration anesthetic sensation that effectively numbs the patient's pain over a period of days with a prolonged reduction in pain thereafter.

The use of low energy acoustic shock waves or pressure pulses that employ a focused beam has been spurred on as a viable alternative to the unfocused low energy shock waves because the focal point being of a small point of energy has little or a small region of cell damage as the remaining portions of the wave pattern can provide a stimulating effect similar to the unfocused shock waves. Basically, the effect is the same with the users of focused waves achieving the benefits of the unfocused waves, but with a focal point of peak energy in a tiny localised region. So, for purposes of the present invention, the use of “soft waves” those defined by low energy beams will be applicable to both focused and unfocused beams o acoustic shock waves or pressure pulses for the present invention.

One last and significant point that the reader must appreciate is that an “acoustic shock wave” is not an “ultrasound wave”. Sonic or ultrasound waves are generated with a uniform and symmetrical wave pattern similar to a sinusoidal wave. This type of sonic wave causes a sheer action on tissue as evidenced by a generation of heat within the tissue, for this reason, the use of sonic waves of the ultrasonic type are not considered as efficient in cell survivability rates. The present invention provides an apparatus for an effective treatment of indications, which benefit from high or low energy pressure pulse/shock waves having focused or unfocused, nearly plane, convergent or even divergent characteristics. With an unfocused wave having nearly plane, plane, convergent wave characteristic or even divergent wave characteristics, the energy density of the wave may be or may be adjusted to be so low that side effects including pain are very minor or even do not exist at all.

In certain embodiments, the apparatus of the present invention is able to produce waves having energy density values that are below 0.1 mJ/mm² or even as low as 0.000 001 mJ/mm². In a preferred embodiment, those low-end values range between 0.1-0.001 mJ/mm². With these low energy densities, side effects are reduced, and the dose application is much more uniform. Additionally, the possibility of harming surface tissue and the fragile sub surface lung tissue is reduced when using an apparatus of the present invention that generates unfocused waves having planar, nearly plane, convergent or divergent characteristics and larger transmission areas compared to apparatuses using a focused shock wave source that need to be moved around to cover the affected area. The apparatus of the present invention also may allow the user to make more precise energy density adjustments than an apparatus generating only focused shock waves, which is generally limited in terms of lowering the energy output. Nevertheless, in some cases the first use of a high energy focused shock wave targeting a treatment zone near the lung tissue but not directly on it may be the best approach followed by a transmission of lower energy unfocused wave patterns directly impinging the lung tissue.

The present invention relates to the use of various therapeutic pressure pulse wave patterns or acoustic shock wave patterns as illustrated in FIGS. 1-3 for treating patients having lung disease or pulmonary disorders that have degraded the respiratory performance. Each illustrated wave pattern will be discussed later in the description; however, the use of each has particularly interesting beneficial features that are a remarkably valuable new tool in the fight against such lung diseases.

With reference to FIGS. 1-3, a variety of schematic views of acoustic shock waves or pressure pulses are described. The following description of the proper amplitude and pressure pulse intensities of the shock waves are provided along with a description of how the shock waves actually function. For the purpose of describing, the shock waves were used as exemplary and are intended to include all of the wave patterns discussed in the figures as possible treatment patterns.

FIG. 1 is a simplified depiction of a pressure pulse/shock wave (PP/SW) generator, such as a shock wave head, showing focusing characteristics of transmitted acoustic pressure pulses. Numeral 1 indicates the position of a generalized pressure pulse generator, which generates the pressure pulse and, via a focusing element, focuses it outside the housing to treat diseases. The affected tissue or organ is generally located in or near the focal point which is located in or near position 6. At position 17 a water cushion or any other kind of exit window for the acoustical energy is located.

FIG. 2 is a simplified depiction of a pressure pulse/shock wave generator, such as a shock wave head, with plane wave characteristics. Numeral 1 indicates the position of a pressure pulse generator according to the present invention, which generates a pressure pulse which is leaving the housing at the position 17, which may be a water cushion or any other kind of exit window. Somewhat even (also referred to herein as “disturbed”) wave characteristics can be generated, in case a paraboloid is used as a reflecting element, with a point source (e.g. electrode) that is located in the focal point of the paraboloid. The waves will be transmitted into the patient's body via a coupling media such as, e.g., ultrasound gel or oil and their amplitudes will be attenuated with increasing distance from the exit window 17.

FIG. 3 is a simplified depiction of a pressure pulse shock wave generator (shock wave head) with divergent wave characteristics. The divergent wave fronts may be leaving the exit window 17 at point 11 where the amplitude of the wave front is very high. This point 17 could be regarded as the source point for the pressure pulses. In Fig 1c the pressure pulse source may be a point source, that is, the pressure pulse may be generated by an electrical discharge of an electrode under water between electrode tips. However, the pressure pulse may also be generated, for example, by an explosion, referred to as a ballistic pressure pulse. The divergent characteristics of the wave front may be a consequence of the mechanical setup.

This apparatus, in certain embodiments, may be adjusted/modified/or the complete shock wave head or part of it may be exchanged so that the desired and/or optimal acoustic profile such as one having wave fronts with focused, planar, nearly plane, convergent or divergent characteristics can be chosen.

A change of the wave front characteristics may, for example, be achieved by changing the distance of the exit acoustic window relative to the reflector, by changing the reflector geometry, by introducing certain lenses or by removing elements such as lenses that modify the waves produced by a pressure pulse/shock wave generating element. Exemplary pressure pulse/shock wave sources that can, for example, be exchanged for each other to allow an apparatus to generate waves having different wave front characteristics are described in detail below.

In one embodiment, mechanical elements that are exchanged to achieve a change in wave front characteristics include the primary pressure pulse generating element, the focusing element, the reflecting element, the housing and the membrane. In another embodiment, the mechanical elements further include a closed fluid volume within the housing in which the pressure pulse is formed and transmitted through the exit window.

In one embodiment, the apparatus of the present invention is used in combinations of shock wave therapies. Here, the characteristics of waves emitted by the apparatus are switched from, for example, focused to divergent or from divergent with lower energy density to divergent with higher energy density. Thus, effects of a pressure pulse treatment can be optimized by using waves having different characteristics and/or energy densities, respectively.

While the above described universal toolbox of the various types of acoustic shock waves or pressure pulses and types of shock wave generating heads provides versatility, the person skilled in the art will appreciate that apparatuses that produce low energy or soft acoustic shock waves or pressure pulses having, for one example, nearly plane characteristics, are less mechanically demanding and fulfill the requirements of many users.

As the person skilled in the art will also appreciate that embodiments shown in the drawings are independent of the generation principle and thus are valid for not only electro-hydraulic shock wave generation but also for, but not limited to, PP/SW generation based on electromagnetic, piezoceramic and ballistic principles. The pressure pulse generators may, in certain embodiments, be equipped with a water cushion that houses water which defines the path of pressure pulse waves that is, through which those waves are transmitted. In a preferred embodiment, a patient is coupled via ultrasound gel or oil to the acoustic exit window (17), which can, for example, be an acoustic transparent membrane, a water cushion, a plastic plate or a metal plate.

FIG. 5 shows an exemplary shock wave device generator or source 1 with a control and power supply 41 connected to a hand-held applicator shock wave head 43 via a flexible hose 42 with fluid conduits. The illustrated shock wave applicator 43 has a flexible membrane at an end of the applicator 43 which transmits the acoustic waves when coupled to the skin by using a fluid or acoustic gel. As shown, this type of applicator 43 has a hydraulic spark generator using either focused or unfocused shock waves, preferably in a low energy level, less than the range of 0.01 mJ/mm² to 0.3 mJ/mm². The flexible hose 42 is connected to a fluid supply that fills the applicator 43 and expands the flexible membrane when filled. Alternatively, a ballistic, piezoelectric or spherical acoustic shock wave device can be used to generate the desired waves.

With reference to FIG. 6, a perspective view of a portion of a treatment region 200 on the back of a patient P is shown between the shoulder and the hip directly. The lungs 100 are the principal source of respiratory activity sending oxygenated red blood cells through the vascular system between the organs and the limbs. With further reference to FIG. 6, the patient P who has lung disease damage or a pulmonary disorder is positioned on a table T preferably face down lying on the stomach. A shock wave applicator head 43 is brought into contact with the skin Ps preferably an acoustic gel is used to enhance the transmission of the shock waves 200 through the body down to the subsurface lung tissue 100 in the region right of the spine 101 below the shoulder preferably at the mid to lower spine region above the lung. The shock wave applicator head 43 is connected via cabling 42 to a power generating unit 41 as shown. The shock wave applicator head 43 can be attached rigidly to a fixture or stand 44 as illustrated or alternatively can be hand held and manipulated across the skin Ps to drive the shock waves 200 in the direction the shock wave head 43 is pointed to activate a response to the lung tissue.

Shock waves are a completely different technology and a quantum leap beyond other forms of respiratory treatments. The mechanism of shock waves is far from being understood, but is known to cause new blood vessels to grow in an area of treatment and regenerate bony tissue. In the present invention shock waves are used to treat patients with lung disease damage or pulmonary disorders by not only regenerating or repairing the lung tissue or creating new lung tissue architecture, but most remarkably reactivating a degraded lung system response. This is a phenomenal advancement in the current approach which generally avoids difficult surgery or can be used in conjunction with a surgically repaired injury as a complimentary treatment to such surgery. If surgery could be replaced in many cases, it would save millions of dollars, gain wide acceptance (non-invasive) and be a tremendous benefit to patients worldwide.

The present invention employs the use of pressure pulses or shock waves to stimulate a tissue or cellular lung response stimulating the respiratory system to respond starting a tissue regenerative healing process that activates the tissue or lung cells not only of damaged cells, but also initiates a systemic healing process to re-energize adjacent affected organs and muscle tissue through an improvement in the degraded respiratory system.

In the pressure pulse or shock wave method of treating a tissue, an organ or the entire body of a human patient with a risk of degenerative neurological or nerve damage or post-occurrence of such damage requires the patient to be positioned in a convenient orientation to permit the source of the emitted waves to most directly send the waves to the target site to initiate pressure pulse or shock wave stimulation of the target area or zone with minimal, preferably with little or no obstructing features in the path of the emitting source or lens. Assuming the treatment region is accessible through an open surgical access region then the shock wave head 43 can be inserted and placed directly on or adjacent to the treatment region 200. Alternatively, the shock wave head 43 can be placed externally on the back, side or frontal chest area and transmit the emitted shock wave patterns through the skin, bone tissue 116 for example and into the underlying lung tissue 100 to be treated, as shown in FIG. 6. In the case of extracorporeal non-invasive treatments of damaged lungs, preferably the outer skin tissue is pressed against the treatment region to insure the transmission loss is minimal. In some cases, the treatment zone may benefit or require numbing prior to treatments in advance of any surgical procedures. This is particularly true after a number of treatments over a period of time, because as the nerves heal, the patient's sensation of pain will be reacquired. This is particularly true if the use of high energy focused waves are being transmitted through the spinal bone tissue to stimulate the sensitive lung tissue in the treatment area. Assuming the target area or site is within a projected area of the wave transmission, a single transmission dosage of wave energy may be used. The transmission dosage can be from a few seconds to 20 minutes or more dependent on the condition. Preferably the waves are generated from an unfocused or focused source. The unfocused waves can be divergent, planar or near planar and having a low pressure amplitude and density in the range of 0.00001 mJ/mm² to 1.0 mJ/mm² or less, most typically below 0.2 mJ/mm². The focused source preferably can use a diffusing lens or have a far-sight focus to minimize if not eliminate having the localized focus point within the tissue. Preferably the focused shock waves are used at a similarly effective low energy transmission or alternatively can be at higher energy but wherein the tissue target site is disposed pre-convergence inward of the geometric focal point of the emitted wave transmission. In treating some hard to penetrate regions, the pressure pulse more preferably is a high energy target focused wave pattern which can effectively penetrate through outer structures prior to being dampened while still exposing the lung tissue to activating pressure pulses or shock waves. This emitted energy preferably stimulates the cells with minimal rupturing of cellular membranes. The surrounding healthy cells in the region treated are activated initiating a defense mechanism response to assist in eradication of the unwanted infection or diseased tissue while stimulating new growth and enhanced autonomic respiratory system performance.

With reference to FIG. 8, a view of a hand of a patient whose reflexology zone 100 is being treated with acoustic shock waves or pressure pulses 200 is illustrated. In this illustration, it is important to note that the applicator 43 presses against the skin Ps of the hand in the reflexology zone 100 for the pancreas which is a region of the right hand in the fatty part below the index finger and a region of the left hand below the middle finger close to the wrist.

FIG. 9 is a perspective view of a foot of a patient whose reflexology zone or target 100 is being treated. A shock wave applicator head 43 is brought into contact with the skin Ps preferably an acoustic gel is used to enhance the transmission of the shock waves 200 through the skin Ps. The shock wave applicator head 43 can be hand held and manipulated across the skin Ps to drive the shock waves 200 in the direction the shock wave head 43 is zoned to activate a stimulating response through the reflexology zone 100. As illustrated, the device shown is an electrohydraulic acoustic shock wave generator, however, other devices that generate acoustic shock waves or pressure pulses can be used. Ultrasonic devices may be considered, but there is no data to support a sinusoidal wave form would work and therefore not considered as effective as the asymmetric wave generators. The acoustic shock waves or pressure pulses activate a cellular response within the reflexology treatment site. This response or stimulation causes an increase of nitric oxide and a release of a variety of growth factors such as VEGF and a release of anti-microbial peptides like LL37. As shown, the flexible membrane is protruding outward and the applicator 43 has been filled with fluid, the transmission or emission of acoustic shock waves or pressure pulses 200 is directed towards the reflexology zone 100. In order to accomplish a good transmission, it is important the flexible membrane be pressed against the patient's skin Ps and as indicated coupling gels may be used. The zone 100, as illustrated, is the reflexology zone for a bone structure which is a region of the foot located along an outside arch of each foot. By transmitting the shock waves 200 to the zone 100, is it believed that a modulation of the pain near the bone structure can be made. This modulation or adjustment can be achieved by transmitting the acoustic waves 200 at low energy directly onto the zone 100. It is believed that a single treatment of the zone 100 will achieve the desired modulation. However, repeated treatments may be administered to help maintain and control this reduced pain level. Having achieved a scheduled pattern of treatments, it is possible to achieve regulation of pain without the use of drugs or other stimulants.

These shock wave energy transmissions are effective in stimulating a cellular response and can be accomplished without creating excessive cavitation bubbles in the tissue of the target site when employed in other than site targeted high energy focused transmissions. This effectively ensures the lung tissue does not have to experience the sensation of tearing or of excessive hemorrhaging so common in the use of higher energy focused wave forms having a focal point at or within the targeted treatment site.

If the target site is the lung subjected to a surgical procedure exposing at least some if not all of the lung tissue, then the target site may be such that the patient or the generating source must be reoriented relative to the site and a second, third or more treatment dosages can be administered. The fact that some if not all of the dosage can be at a low energy the common problem of localized hemorrhaging can be reduced making it more practical to administer multiple dosages of waves from various orientations to further optimize the treatment and cellular stimulation of the target site. Heretofore focused high energy multiple treatments induced pain and discomfort to the patient. The use of low energy focused or un-focused waves at the target site enables multiple sequential treatments with minimal pain.

The present method may need precise site location and can be used in combination with such known devices as ultrasound, cat-scan or x-ray imaging if needed. The physician's general understanding of the anatomy of the patient may be sufficient to locate the target area to be treated. This is particularly true when the exposed lung tissue or portion of the trauma to the body or organ is visually within the surgeon's line of sight and this permits the lens or cover of the emitting shock wave source to impinge on the affected organ or tissue directly or through a transmission enhancing gel, water or fluid medium during the pressure pulse or shock wave treatment. The treated area can withstand a far greater number of shock waves based on the selected energy level being emitted. For example at very low energy levels the stimulation exposure can be provided over prolonged periods as much as 20 minutes if so desired. At higher energy levels the treatment duration can be shortened to less than a minute, less than a second if so desired. The limiting factor in the selected treatment dosage is minimization of surrounding cell hemorrhaging and other kinds of damage to the surrounding cells or tissue while still providing a stimulating stem cell activation or a cellular release or activation of proteins such as brain derived neurotropic factor (BDNF) or VEGF and other growth factors while simultaneously germicidally attacking the degenerative tissue or infectious bacteria at the wound site.

Due to the wide range of beneficial treatments available it is believed preferable that the optimal use of one or more wave generators or sources should be selected on the basis of the specific application. Wherein relatively small target sites may involve a single wave generator placed on an adjustable manipulator arm. A key advantage of the present inventive methodology is that it is complimentary to conventional medical procedures. In the case of any operative surgical procedure the surgical area of the patient can be bombarded with these energy waves to stimulate cellular release of healing agents and growth factors. This will dramatically reduce the healing process time. Most preferably such patients may be provided more than one such treatment with an intervening dwell time for cellular relaxation prior to secondary and tertiary post-operative treatments.

The underlying principle of these pressure pulse or shock wave therapy methods is to enrich the treatment area directly and to stimulate the body's own natural healing capability by causing the degraded respiratory system to activate a response. This is accomplished by deploying shock waves to stimulate strong cells in the lung tissue and the surrounding tissue to activate a variety of responses. The acoustic shock waves transmit or trigger what appears to be a cellular communication throughout the entire anatomical structure, this activates a generalized cellular response at the treatment site, in particular, but more interestingly a systemic response in areas more removed from the wave form pattern. This is believed to be one of the reasons molecular stimulation can be conducted at threshold energies heretofore believed to be well below those commonly accepted as required. Accordingly, not only can the energy intensity be reduced in some cases, but also the number of applied shock wave impulses can be lowered from several thousand to as few as one or more pulses and still yield a beneficial stimulating response. The key is to provide at least a sufficient amount of energy to activate healing reactions.

Even more striking as mentioned earlier, early prevention therapies can be employed to stimulate tissue or organ modelling to be maintained within acceptable ranges prior to an exposure to a degenerative failure. This is extremely valuable in the prevention of spreading the infection or degenerative condition for example. The methods would be to identify at risk patients with a known exposure risk and subjecting that patient to therapeutic shock wave therapy for the purpose of stimulating lung tissue repair or regeneration effectively remodelling the patient's susceptible lungs to be within accepted functional parameters prior to irreparable degeneration. The objective being to preventively stimulate cellular tissue repairs to pre-emptively avoid a degenerative condition from occurring which may result in the onset of a degenerative condition which can require invasive surgical procedures.

As shown in FIGS. 1-3 the use of these various acoustic shock wave forms can be used separately or in combination to achieve the desired therapeutic effect in treating patients with lung tissue damage, most importantly to trigger an entire respiratory system response in a degraded respiratory system caused by an injury or lesions.

Furthermore, such acoustic shock wave forms can be used in combination with drugs, chemical treatments, irradiation therapy or even physical therapy and when so combined the stimulated cells will more rapidly assist the body's natural healing response and thus overcomes the otherwise potentially tissue damaging effects of these complimentary procedures.

The present invention provides an apparatus for an effective treatment of indications, which benefit from high or low energy pressure pulse/shock waves having focused or unfocused, nearly plane, convergent or even divergent characteristics. With an unfocused wave having nearly plane, plane, convergent wave characteristic or even divergent wave characteristics, the energy density of the wave may be or may be adjusted to be so low that side effects including pain are very minor or even do not exist at all.

In one preferred embodiment, the direct treatment of the lungs can be enhanced by the use of a separate shock wave or pressure pulse treatment directed to a reflexology region of an extremity of a limb such as the hand or foot as illustrated in FIGS. 8 and 9. As shown, a reflexology zone for the respiratory system is treated with the pressure pulses or shock waves to stimulate a healing response of the diseased lungs. This combination has had extraordinary success in treating patients with COPD bringing the patients lung performance back to normal after a few treatments. The techniques of treating reflexology regions is best described in U.S. application Ser. No. 16/353,365 filed Mar. 14, 2019, entitled, “Acoustic Shock Wave Therapeutic Methods To Treat Medical Conditions Using Reflexology Zones”, which is being incorporated by reference herein in its entirety. An important aspect in treating the respiratory system is the use of pressure pulses or shock waves is excellent at reducing all sources of inflammation. This ability to reduce inflammation greatly helps in curing disease wherein there is noticeable swelling and redness of inflamed cells. Accordingly, the diseases of bronchitis, asthma, COPD and all those types of lung diseases can be most beneficially treated.

In addition to treating disorders of the lung, there are numerous individuals that can greatly benefit by increasing their lung capacity. For example, long distance runners, divers, military personnel, and firefighters all require improved, highly efficient lungs. The present methods provide a way to increase the lung's capacity and to improve oxygenation of the blood supply.

The inventors of the present invention have noticed that competitors wishing to circumvent the intellectual property rights of their patented work have developed creative language to appear as though their methods are somehow different from the teachings employed herein. To clarify this situation, applicants want to explain that the energy of the pressure pulses or shock waves is to be defined at the interface of the device membrane or exit window and the patient's skin, this interface is typically acoustically coupled to the device suing an acoustic gel. It is at this location where the energy density is to be measured. At this location, the wave pattern has left the device and enters the patient. In radial type devices or spherical wave generators, the wave pattern emanates radially from a single source. In a fluid filled balloon using a spark generator, the energy at the gap in the electrodes can be extremely high 10 mJ/mm2 at 20 kV, however, at the membrane wall coupled to the patient's skin, the energy transmitted to the patient is less than 1 mJ/mm2 typically 0.2 or less. In ballistic type devices, the sound wave energy at the exit window is at a peak that drops off as it enters the tissue. Accordingly, the most reasonable and convenient way to measure the energy is at the interface with it being understood the wave pulse energy lessens as it travels through the tissue and bone structure. Some recent articles have suggested that the transmission of the sound wave is altered as the wave pattern penetrates through bone converting the acoustic shock wave to an ultrasonic wave. In a paper entitled, “Transcranial Pulse Stimulation with Ultrasound in Alzheimer's Disease—A New Navigated Focal Brain Therapy”, by R. Beisteiner et al, a Storz shock wave device is employed, but the authors suggest the shock wave is transformed to a brain stimulating ultrasonic pulse. This revelation, if true, ignores the fact the device at the membrane or exit window is transmitting shock waves at the interface of the skin. The authors observation as to the energy at the brain tissue ignores the fact the treatment is an acoustic shock wave treatment patented by the inventors of the present invention. Once the shock wave leaves the exit window, there is no control of the wave characteristic as it penetrates bone and tissue. Accordingly, the paper, while interesting, is more of an explanation that an invention. Years earlier, in U.S. Pat. No. 7,544,171 issued Jun.9, 2009, the present inventor, Warlick, was granted patent rights to the treatment of dementia and Alzheimer's. The point of this discussion is the recent manipulation of how one measures or defines the way the acoustic or sound energy reaches the tissue circumvents the truly inventive work of low energy pressure pulses or shock waves. The present invention encompasses all asymmetrical waves or pulses at low energy as defined herein.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A method of treating a patient exhibiting a lung disease or pulmonary disorder by applying shock waves or acoustic pulses directed to impinge lung tissue of the lung or lungs exhibiting a lung disease or pulmonary disorder, comprises the steps of:
activating an acoustic shock wave or acoustic wave generator or source to emit acoustic shock waves or pressure pulses from a fixed acoustic wave source or a handheld shock wave or pressure pulse head; and
administering a plurality of acoustic waves in a pressure pulse or shock wave pattern from an exit window or membrane of the fixed acoustic wave source or a handheld shock wave or pressure pulse head coupled to the patient's skin of less than 10.0 mJ/mm² per shock wave or pressure pulse toward the lung tissue, the plurality of acoustic waves in a pressure pulse or shock wave pattern being directed to a portion of the lung exhibiting the lung disease or pulmonary disorder.

2. The method of claim 1 wherein the step of administering a plurality of acoustic waves delivered as shock waves or pressure pulses to the lung further reduces symptoms of the lung disease or pulmonary disorder.

3. The method of claim 1 wherein the lung disease or pulmonary disorder is one of asthma, bronchitis, Chronic Obstructive Pulmonary Disease (COPD), cystic fibrosis, emphysema, Idiopathic pulmonary fibrosis (IPF), flu, lung cancer, obstructive sleep apnea, pleurisy, pneumonia, or tuberculosis (TB).

4. The method of claim 1 wherein the treatment further comprises administering acoustic shock waves or pressure pulses directed to an area of the lung, or to a reflexology zone to treat the lung disease or pulmonary disorder.

5. The method of claim 1 wherein the reflexology zone is at an extremity of a limb.

6. The method of claim 1 wherein the extremity is a hand or foot.

7. The method of claim 1 wherein the plurality of acoustic waves in the pressure pulse or shock wave pattern from the exit window or membrane of the fixed acoustic wave source or handheld shock wave or pressure pulse head coupled to the patient's skin are less than 1.0 mJ/mm² per shock wave or pressure pulse.

8. The method of claim 1 wherein the acoustic shockwave or acoustic wave generator or source or handheld applicator or fixed applicator source is a spherical device.

9. The method of claim 1 wherein the acoustic shockwave or acoustic wave generator or source or handheld applicator or fixed applicator source is a ballistic device.

10. The method of claim 1 wherein the acoustic shockwave or acoustic wave generator or source or handheld applicator or fixed applicator source is a radial device.

11. The method of claim 1 wherein the acoustic shockwave or acoustic wave generator or source or handheld applicator or fixed applicator source is an electrohydraulic device.

12. The method of claim 1 wherein the acoustic shockwave or acoustic wave generator or source or handheld applicator or fixed applicator source is a piezoelectric device.

13. The method of claim 1 wherein the acoustic shockwave or acoustic wave generator or source or handheld applicator or fixed applicator source is a laser device.

14. The method of claim 1 wherein the acoustic shockwave or acoustic wave generator or source or handheld applicator or fixed applicator source is an electromagnetic device.

15. The method of claim 1 wherein the acoustic shockwave or acoustic wave generator or source or handheld applicator or fixed applicator source is an ultrasound device.

16. The method of claim 1 wherein the acoustic shockwave or acoustic wave generator or source or handheld applicator or fixed applicator source is a hybrid ultrasound device.

17. The method of claim 1 wherein the acoustic shockwave or acoustic wave generator or source or handheld applicator or fixed applicator source is a pulsed wave device.

18. The method of claim 1 wherein the acoustic shockwave or acoustic wave generator or source or handheld applicator or fixed applicator source is a continuous wave device.

19. A method to improve lung capacity by applying shock waves or acoustic pulses or continuous waves directed to tissue of the lung or lungs, comprises the steps of:
activating an acoustic shock wave or acoustic wave generator or source to emit acoustic shock waves or pressure pulses from a fixed acoustic wave source or a handheld shock wave or pressure pulse head; and
administering a plurality of acoustic waves in a pressure pulse or shock wave pattern from an exit window or membrane of the fixed acoustic wave source or a handheld shock wave or pressure pulse head coupled to the patient's skin of less than 10.0 mJ/mm2 per shock wave or pressure pulse toward the lung tissue, the plurality of acoustic waves in a pressure pulse or shock wave pattern being directed to a portion of the lung.
